(19) United States
(12) Patent Application Publication
(10) Pub. No.: US 2020/0024563 A1
(43) Pub. Date: Jan. 23, 2020

(54) METHOD AND SYSTEM FOR A CELL CULTURE SYSTEM WITH RECIRCULATING CULTURE MEDIUM

(71) Applicant: Waterloo Instruments Inc., Waterloo (CA)

(72) Inventors: Caleb HORST, Waterloo (CA); Jim VELDHUIS, Waterloo (CA)

(21) Appl. No.: 16/516,825

(22) Filed: Jul. 19, 2019

Related U.S. Application Data
(60) Provisional application No. 62/700,951, filed on Jul. 20, 2018.

(51) Int. Cl. C12M 3/06; C12M 1/00; B01L 3/00

(52) U.S. Cl. CPC C12M 27/16; C12M 29/04; B01L 2400/0457; B01L 2300/044; B01L 2300/0877; B01L 3/502

(57) ABSTRACT

An apparatus for culturing cells without pumps or moving parts is described. The apparatus includes a supply reservoir and an effluent reservoir that are connected via a cell culture chamber for hosting cells and a bypass channel. The cell culture chamber and the bypass channel are fluidically coupled between the supply reservoir and the effluent reservoir. When the apparatus is in a first position, fluid held in the supply reservoir flows to the effluent reservoir via the cell culture chamber and when the apparatus is in a second position, fluid flows from the effluent reservoir to the supply reservoir via the bypass channel.

### CROSS-REFERENCE TO OTHER APPLICATIONS

This application claims priority from U.S. Provisional Application No. 62/700,951 filed Jul. 20, 2018, the contents of which are hereby incorporated by reference.

### FIELD

The current disclosure is generally directed at an in vitro cell culture system and, more specifically, at a method and system for a cell culture system with recirculating culture medium.

### BACKGROUND

Current cell culture systems involve cells immersed in a static volume of liquid media. This differs from the in vivo environment, where liquid media, such as blood, flows through the system to exchange nutrients, gases, and waste products with the nearby cells. There currently exist systems that simulate physiological fluid, or liquid media, flow in several manners. At a large scale, fluid is circulated using pumps and controlled by valves. To achieve independent small-scale cultures, micropumps, reciprocating gravity-flow, or closed channel microfluidic circuits are used to provide fluid flow.

In general, cells are cultured in static fluid, non-recirculating flow, reciprocating flow, or recirculating flow conditions. Static fluid cell culture systems involve culturing the cells while they are submerged in a volume of liquid media. This is the simplest method of cell culture as there is little or no need to continuously move the fluid. Non-recirculating flow cell culture systems involve moving fluid past the cultured cells in one or more culture regions from a supply reservoir to an effluent reservoir. A non-recirculating flow cell culture system can be simple as there is no need to recapture and re-pump the fluid. However, in many cases, this type of system is undesirable as large volumes of fluid are required to sustain cell cultures over time. Reciprocating flow cell culture systems are similar to non-recirculating flow cell culture systems except that the flow is periodically reversed. These systems use fluid efficiently, but do not recapitulate unidirectional physiological conditions and in some cases this may have a negative impact on the cultured cells. In general, a recirculating flow cell culture system involves a flow loop that continuously or intermittently moves fluid past the cultured cells in one of more culture regions without any loss of media or reversal of the flow direction.

Therefore, there is provided a novel method and system for a cell culture system with recirculating culture medium.

### SUMMARY

In one aspect of the disclosure, there is provided an apparatus for culturing cells including a supply reservoir; an effluent reservoir; a cell culture chamber for hosting cells, the cell culture chamber fluidically coupled between the supply reservoir and the effluent reservoir; and a bypass channel fluidically coupled between the supply reservoir and the effluent reservoir; wherein when the apparatus is in a first position, fluid held in the supply reservoir flows to the effluent reservoir via the cell culture chamber and when the apparatus is in a second position, fluid flows from the effluent reservoir to the supply reservoir via the bypass channel.

In another aspect, the supply reservoir, the effluent reservoir, the cell culture chamber and the bypass chamber as formed by a single piece of material. In a further aspect, the single piece of material is rigid and porous. In yet a further aspect, the single piece of material is metal, ceramic, plastic or glass.

In another aspect, in the second position, fluid flows from the effluent reservoir to the supply reservoir via the cell culture chamber. In an aspect, the bypass channel is coupled to the supply reservoir at a supply reservoir and bypass channel coupling position higher than the fluid level when the apparatus is in the first position. In a further aspect, the system further includes a recirculation barrier to define a height of the supply reservoir and bypass channel coupling position. In yet another aspect, the bypass channel is coupled to the effluent reservoir at a effluent reservoir and bypass channel coupling position lower than the supply reservoir and bypass channel coupling position. In yet a further aspect, the cell culture chamber is coupled to the supply reservoir and the effluent reservoir at positions beneath the supply reservoir and bypass channel coupling position and effluent reservoir and bypass channel coupling position.

In an aspect, the cell culture chamber is positioned below the supply reservoir and below the effluent reservoir. In another aspect, the cell culture chamber includes at least two channels. In yet a further aspect, the at least two channels are parallel to each other. In another aspect, the at least two channels are connected via a porous membrane. In yet another aspect, the cell culture chamber includes at least two chambers. In another aspect, the at least two chambers are in series with each other. In another aspect, the cell culture chamber is remote from the supply reservoir and the effluent reservoir.

In another aspect of the disclosure, there is provided a system for culturing cells including a set of cell culture apparatus, each of the cell culture apparatus including: a supply reservoir; an effluent reservoir; cell culture chamber for hosting cells, the cell culture chamber fluidically coupled between the supply reservoir and the effluent reservoir; and a bypass channel fluidically coupled between the supply reservoir and the effluent reservoir; wherein when the system is in a first position, fluid held in the supply reservoir of each of the set of cell culture apparatus flows to the effluent reservoir via the cell culture chamber and when the system is in a second position, fluid flows from the effluent reservoir of each of the cull culture apparatus to the supply reservoir via the bypass channel.

In another aspect, the set of cell culture apparatus are spatially segregated from each other. In yet another aspect, the set of cell culture apparatus are interleaved with each other. In yet a further aspect, the set of cell culture apparatus is formed by single piece of material.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 is a partial cutaway view of a first embodiment of a cell culture apparatus;

FIG. 2 is a cutaway view of the cell culture apparatus of FIG. 1;

FIG. 3 is a top perspective view of the cell culture apparatus of FIG. 1;

FIG. 4A is a schematic view of a cell culture apparatus containing a fluid media that has been tilted to a first position at a beginning of forward flow;

FIG. 4B is a schematic view of the cell culture apparatus of FIG. 4a containing a fluid media at an end of forward flow;

FIG. 4C is a schematic view of the cell culture apparatus of FIG. 4a containing a fluid media that has been tilted to a second position at a beginning of return flow;

FIG. 4D is a schematic view of the cell culture apparatus of FIG. 4a containing a fluid media near an end of return flow;

FIG. 4E is a schematic view of the cell culture apparatus of FIG. 4a in a level position illustrating the difference in height of the fluid levels that can be used to drive fluid flow;

FIG. 5 is a perspective view of another embodiment of a cell culture apparatus;

FIG. 6 is a perspective view of a further embodiment of a cell culture apparatus;

FIG. 7 is a partial cutaway orthogonal view of segment of the cell culture apparatus;

FIG. 8 is a top perspective view of the cell culture apparatus of FIG. 7;

FIG. 9 is a partial cutaway bottom perspective view of the cell culture apparatus of FIG. 7;

FIG. 10 is a partial cutaway bottom perspective view of the cell culture apparatus of FIG. 7;

FIG. 11A is a cross-sectional diagram of a cell culture system at the beginning of forward flow;

FIG. 11B is a cross-sectional diagram of the cell culture apparatus at the end of forward flow;

FIG. 11C is a cross-sectional diagram of the cell culture apparatus at the end of return flow

FIG. 11D is a cross-sectional diagram of the cell culture apparatus in an initial forward flow position;

FIG. 11E is a cross-sectional diagram of the cell culture apparatus at the beginning of return flow.

FIG. 12 shows a flow diagram for a method of recirculating fluid media through a cell culture chamber;

FIG. 13 is a schematic diagram of another embodiment of a cell culture apparatus;

FIG. 14 is a schematic diagram of another embodiment of a cell culture apparatus;

FIG. 15 is a schematic diagram of another embodiment of a cell culture apparatus;

FIG. 16 is a schematic diagram of another embodiment of a cell culture apparatus; and

FIG. 17 is a schematic diagram of another embodiment of a cell culture apparatus with a tilting mechanism.

### DETAILED DESCRIPTION OF THE DISCLOSURE

The disclosure is directed at a method and system for a cell culture system with recirculating culture medium. In the following description, the term culture medium may also be referred to as fluid, fluid media, or liquid media.

In one embodiment, the disclosure includes a system, apparatus and method that generates recirculating fluid flow through a cell culture chamber containing cultured cells that may be adhered to the walls of the cell culture chamber. The cell culture chamber may contain a three-dimensional (3D) porous material with cultured cells distributed within the material, and the flow may be delivered such that the fluid flows through the 3D porous material. The presence of the recirculating fluid flow benefits the cultured cells in the cell culture chamber by delivering nutrients, removing waste products, and exchanging gases. The presence of the fluid flow may also benefit the cultured cells by providing fluid shear to the cells. The presence of fluid flow and the control of flow rate patterns may also effect other changes in the cultured cells such as, but not limited to, differentiation, gene expression, protein production, cell alignment, and other cellular activities and processes.

In one embodiment, the disclosure includes a bypass channel that connects supply and effluent reservoirs. The bypass channel may be physically oriented such that no fluid can flow within the bypass channel when the apparatus is in a first or forward flow position that creates or generates normal (forward) fluid, or fluid media, flow through the cell culture chamber. In contrast, when the apparatus is in a second, or return flow position, the physical orientation of the bypass channel allows fluid flow from the effluent reservoir to the supply reservoir.

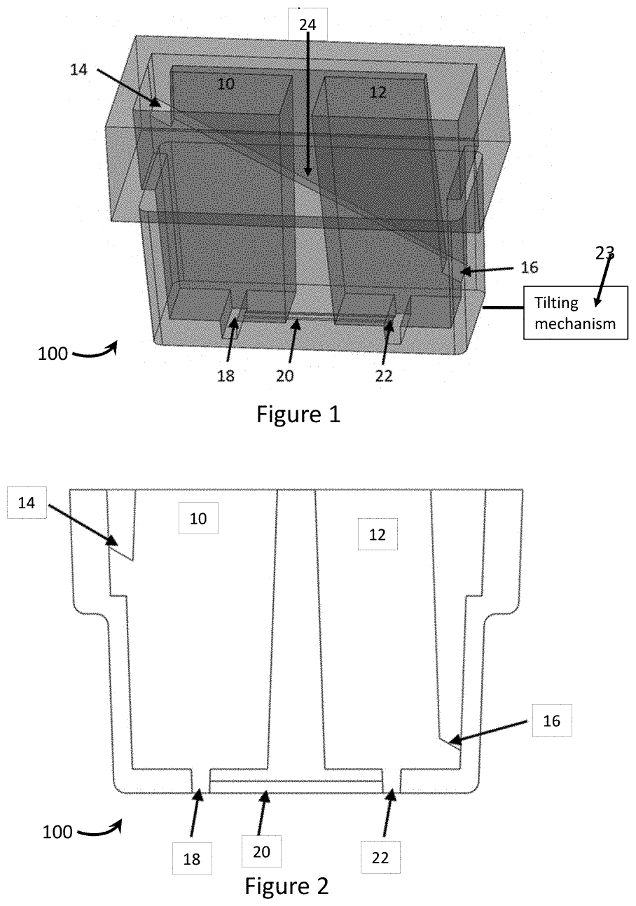

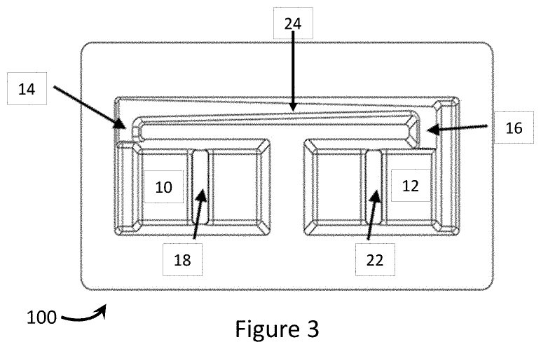

Turning to FIG. 1, a somewhat transparent perspective view of a cell culture system or apparatus is shown. FIG. 2 is a partial cutaway from view of the cell culture apparatus and FIG. 3 is a top view of the cell culture apparatus. In the current embodiment, cell culture apparatus 100 includes a supply reservoir 10, an effluent reservoir 12, a cell culture chamber 20, and a bypass channel 24.

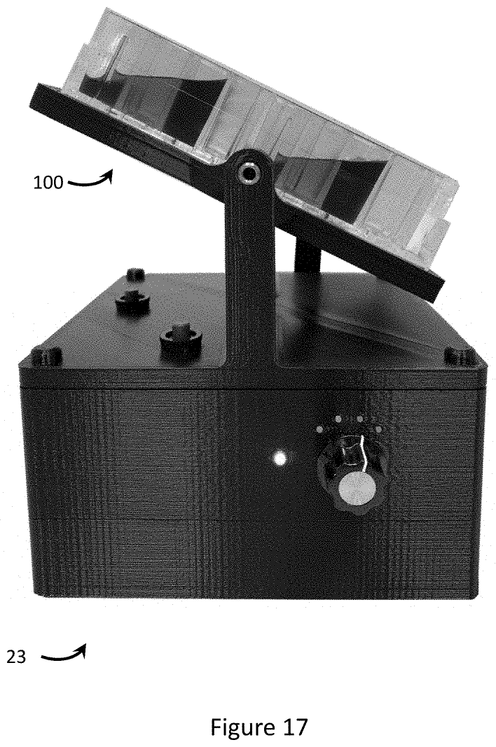

The supply reservoir 10 and the effluent reservoir 12 are fluidly connected by both the bypass channel 24 and the cell culture chamber 20. In a preferred embodiment, the supply reservoir 10 is connected to the cell culture chamber 20 via a cell culture channel entrance 18 and to the bypass channel 24 via a bypass channel exit 14. The effluent reservoir 12 is connected to the cell culture chamber 20 via a cell culture chamber exit 22 and to the bypass channel 24 via a bypass channel entrance 16. In a preferred embodiment, a size, such as the cross-section or volume of the bypass channel 24 is larger than the cross-section or volume of the cell culture chamber 20 such that the bypass channel 24 can support higher fluid flow rates than the cell culture chamber 20. The cell culture apparatus 100 may further include a tilting mechanism 23 for moving the apparatus 100 between different positions, namely the forward flow and return flow positions. As will be understood, the apparatus 100 may also be placed in other positions between or past the forward flow and return flow positions. Examples of a tilting mechanism include, but are not limited to, mechanical actuators include a tiltable table, a tiltable arm, and/or a robotic arm. Actuation may be achieved with a linear motor or a rotating actuator, either of which may be electrical, pneumatic, hydraulic, or mechanical. One embodiment of the apparatus 100 connected to a tilting mechanism 23 is shown in FIG. 17.

In one embodiment, the cell culture apparatus 100 is constructed such that fluid flow, i.e. the flow of a fluid media between the reservoirs, is driven by gravity by tilting the apparatus one way or another. While this may be performed by the tilting mechanism 23, this may also be performed manually. Throughout this specification, the term “forward flow” is defined as flow from the supply reservoir to the effluent reservoir via the cell culture chamber, the term “return flow” is defined as flow from the effluent reservoir to the supply reservoir via the bypass channel, and the term “backflow” is defined as flow from the effluent reservoir to the supply reservoir via the cell culture chamber.

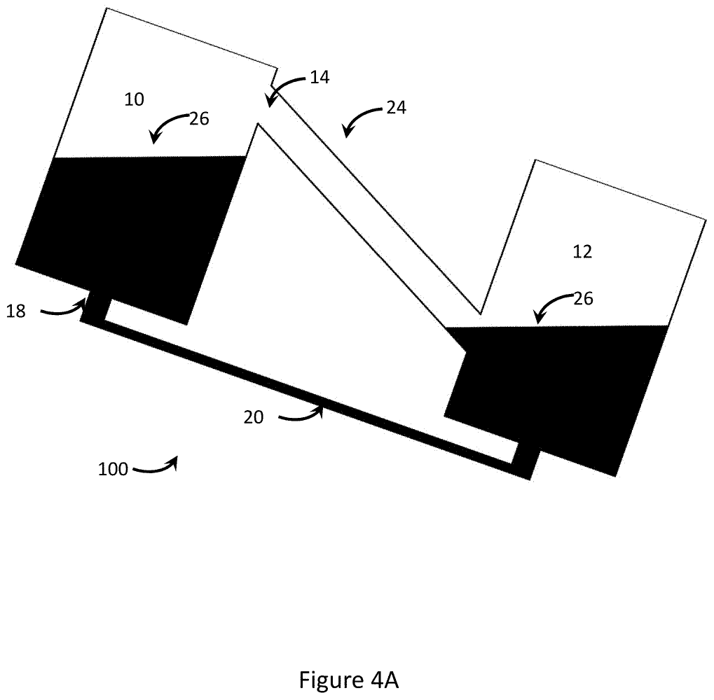

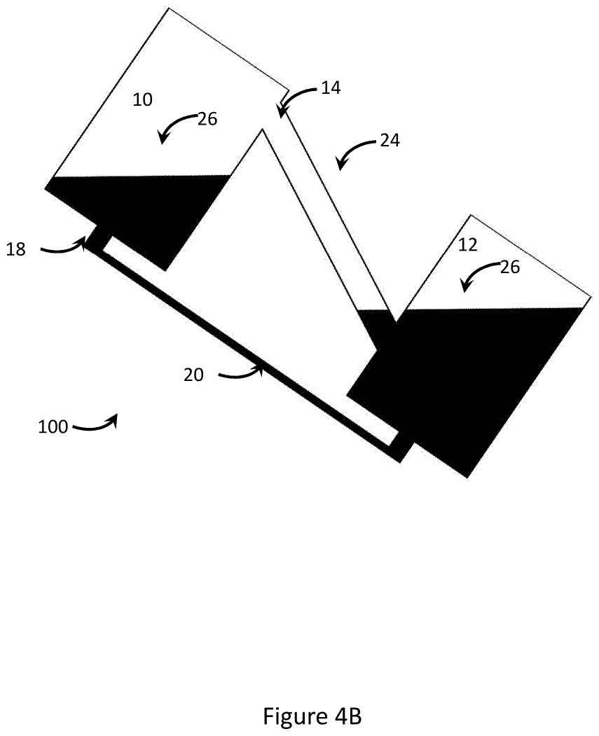

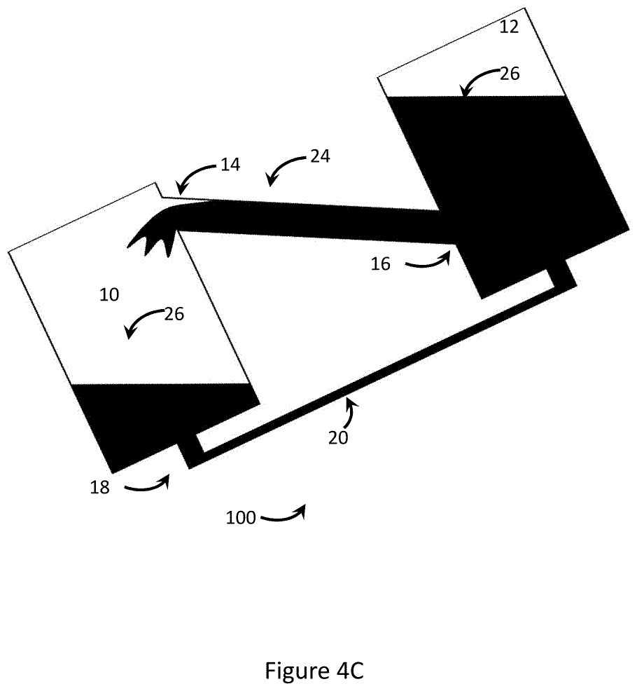

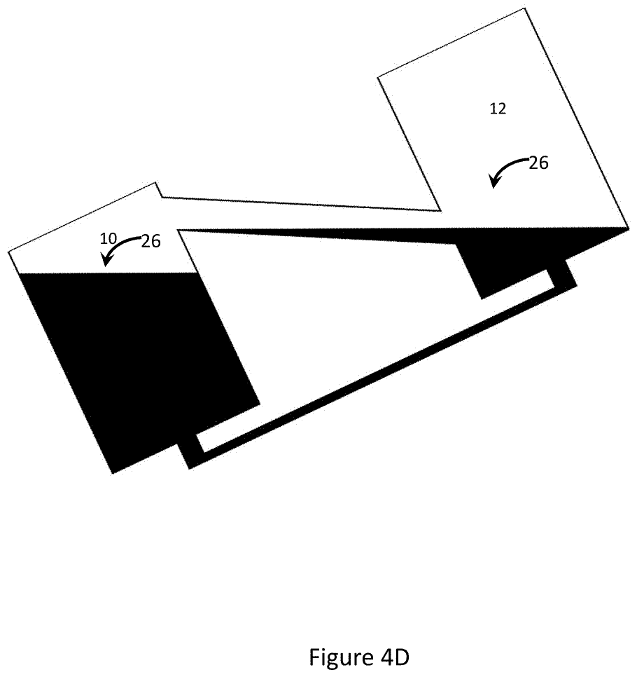

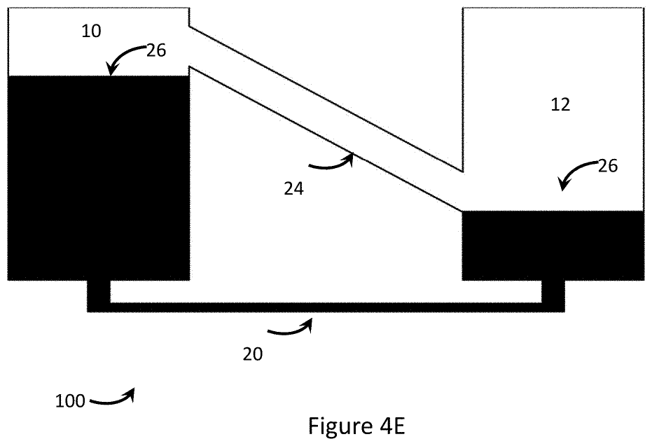

FIG. 4A is a schematic view of a cell culture apparatus 100 in the first, or forward flow, position. As can be seen, the cell culture apparatus 100 includes a fluid media 26 that is passing or flowing from the supply reservoir 10 to the effluent reservoir 12 via the cell culture chamber 20. In other words, the apparatus 100 has been tilted to the first position representing a beginning of forward flow. The cell culture apparatus 100 is tilted to the first position by tilting the cell culture apparatus (such as from a position as shown in FIG. 4E) in a first direction, for example in a clockwise direction. Tilting the cell culture apparatus 100 to the first position raises the position of the supply reservoir 10 relative to the effluent reservoir 12 thereby creating a static pressure head in supply reservoir 10 that drives fluid flow from supply reservoir 10 to effluent reservoir 12. The static pressure head in supply reservoir 10 is due to the level of the fluid media 26 in the supply reservoir 10 being higher than the level of the fluid media 26 in the effluent reservoir 12. As the bypass channel exit 14 is higher than the culture channel entrance 18, the fluid media 26 flows to the effluent reservoir 12 via the cell culture channel 20 rather than via the bypass channel 24.

In general, the system, or apparatus may be seen as being in a plurality of states. It will be understood that these states may correspond with specific positions as described herein but that the system, or apparatus, will also be in multiple positions between the specific positions and/or plurality of states.

State 1 may be seen as a full supply reservoir and empty effluent reservoir state with the supply reservoir at its lowest point with respect to a position of the effluent reservoir. In State 1 the apparatus is in the second position. State 2 may be seen as the stage where there is a development of a pressure head within the supply reservoir which starts the flow of fluid from the supply reservoir to the effluent reservoir. In State 2 the apparatus is in an intermediate position. This state may be associated with the forward flow position. State 3 may be seen as the full effluent reservoir state with the supply reservoir higher than the effluent reservoir. In other words, where the apparatus has been “fully” tilted such that the supply reservoir is at its highest point with respect to the effluent reservoir. In State 3 the apparatus is in the first position. State 4 may be seen as the empty supply reservoir and full effluent reservoir where the effluent reservoir is higher than the supply reservoir. In other words, the apparatus has been “fully” tilted such that the effluent reservoir is at its highest point with respect to the supply reservoir. This may be associated with the reverse flow position. In State 4 the apparatus is in the second position. As the fluid flows from the effluent reservoir to the supply reservoir via at least the bypass channel, the apparatus returns to State 1 where the process is repeated.

If the fluid level in supply reservoir 10 is below the bypass channel exit 14 at the beginning of forward flow, the fluid media 26 may flow to the effluent reservoir 12 solely via cell culture chamber 20. The flow of fluid media 26 from the supply reservoir 10 to the effluent reservoir 12 lowers the fluid level of the fluid media 26 in supply reservoir 10 and raises the fluid level of the fluid media 26 in the effluent reservoir 12, thereby decreasing the pressure head in supply reservoir 10. Eventually, the fluid level of the fluid media 26 in supply reservoir 10 and the fluid level of the fluid media 26 in the effluent reservoir 12 equal and forward flow ceases. If the fluid level in supply reservoir 10 is above the bypass channel exit 14 at the beginning of forward flow, the fluid media 26 may flow to the effluent reservoir 12 via the bypass channel 24 and the cell culture chamber 20 until the fluid level of the fluid media in the supply reservoir 10 falls below the bypass channel exit 14, at which point the fluid media 26 will flow to the effluent reservoir 12 via the cell culture chamber 20.

FIG. 4B is a schematic view of the cell culture apparatus 100 containing a fluid media 26 at an end of forward flow. In a preferred embodiment, the tiling mechanism continually tilts the apparatus back and forth (i.e. in a counter clockwise direction and then a clockwise direction in a repeated manner). In one embodiment, the comparison of fluid levels is between the highest level of the supply reservoir 10 and the lowest level of the effluent reservoir 12. As can be seen, there is a greater amount of fluid media 26 present in effluent reservoir 12 compared to the supply reservoir 10. The cell culture apparatus 100 may then be tilted to or towards the second, or return flow, position to begin return flow.

FIG. 4C is a schematic view of the cell culture apparatus 100 that has been tilted to the second position representing a beginning of return flow. The cell culture apparatus 100 may be tilted to the second position by tilting the cell culture apparatus in a direction opposite the first direction, for example in a counter-clockwise direction. Tilting the cell culture apparatus 100 to or towards, the second position raises the position of effluent reservoir 12 relative to the position of supply reservoir 10 creating a static pressure head in effluent reservoir 12 to drive fluid flow from the effluent reservoir 12 to the supply reservoir 10.

In the current embodiment, the bypass channel entrance 16 is below the fluid level of the fluid media 26 in the effluent reservoir 12, which allows fluid media 26 to flow through the bypass channel 24. If the bypass channel 24 is larger than the cell culture chamber 20 (as in a preferred embodiment), the flow rate of fluid media through the bypass channel 24 is higher than the flow rate through the cell culture chamber 20. In other words, the rate of return flow through the bypass channel is higher than the rate of back-flow through the cell culture chamber. Over time, the flow of the fluid media 26 from the effluent reservoir 12 to the supply reservoir 10 cause the fluid levels in the reservoirs to equalize and the return flow will slow and eventually stop if no further tilting takes place.

FIG. 4D is a schematic view of the cell culture apparatus 100 containing a fluid media 26 nearing the end of return flow. There is a greater amount of fluid media 26 present in supply reservoir 10 compared to effluent reservoir 12, however the angle by which cell culture apparatus 100 is tilted causes the fluid flow through the bypass channel to stop. The cell culture apparatus may be tilted back to the first position to begin forward flow to restart the fluid flow cycle.

As discussed above, if the physical dimensions of the bypass channel 24 are larger than the cell culture chamber 20, the time required for return flow to be completed may be less than the time required for forward flow to be completed. If the time required for return flow is very small relative to the time required for forward flow, the cells in the cell culture chamber 20 may be exposed to forward flow nearly all the time. In one embodiment, the disclosure may be seen as being directed at a recirculating fluid flow system for a cell culture chamber. Recirculating flow creates a flow loop where the fluid is pumped back to the supply reservoir via a secondary pathway, or bypass channel, and then resupplied to the cell culture. In many cases recirculating flow provides a cell culture environment that is similar to in vivo conditions while at the same time making efficient use of media.

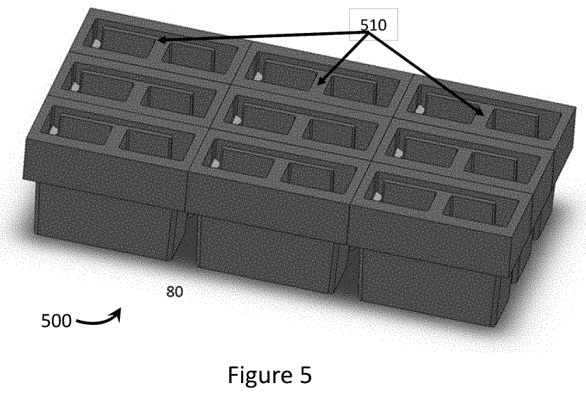

FIG. 5 is a perspective view of a cell culture system 500 in accordance with the present disclosure. The cell culture system 500 includes a plurality of cell culture apparatus 100, or recirculation loops, 510 physically coupled to one another. Each of the plurality of recirculation loops 510 may be substantively similar to cell culture apparatus 100. Tilting of cell culture system 500 tilts each of the plurality of recirculation loops 510, thereby recirculating fluid media through each of the recirculation loops 510, such as described above with respect to the cell culture apparatus 100.

For each recirculation loop 510 (or the cell culture apparatus 100 of FIG. 1), the reservoirs, channels can all be molded from a small number of rigid components with no internally moving parts. This may also include a single piece of material. In a preferred embodiment, the flow conditions in each recirculation loop 510 within the system 500 are preferably identical and controlled by a single mechanical actuator (or tilting mechanism) capable of tilting the entire system 500 in a clockwise and counter-clockwise in a controlled manner.

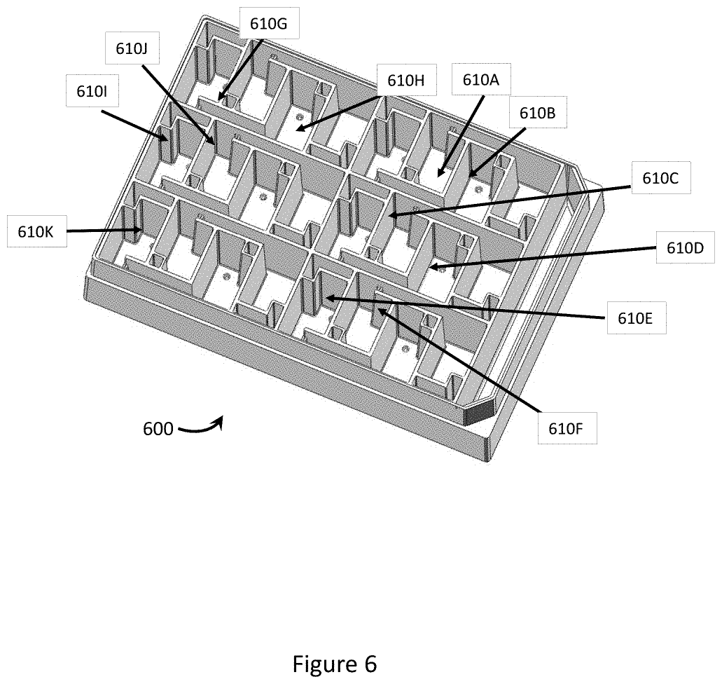

FIG. 6 is a perspective view of a cell culture system 600 in accordance with the present disclosure. The cell culture system 600 includes twelve recirculation loops 610A-L, referred to collectively as the recirculation loops 610. While the current embodiment includes 12 recirculation loops, in alternative embodiments a cell culture system in accordance with the present disclosure may contain fewer or more recirculation loops. The cell culture system 600 is similar in some ways to the cell culture system 500 with one difference being that while the recirculation loops 510 of cell culture system 500 are completely spatially segregated from one another, the recirculation loops 610 of cell culture system 600 are interleaved with one another. Interleaving the recirculation loops increases the number of recirculation loops that may fit into a given amount of space, increasing the number of recirculation loops that may fit into a single system and reducing the amount of material necessary to manufacture cell culture system 600.

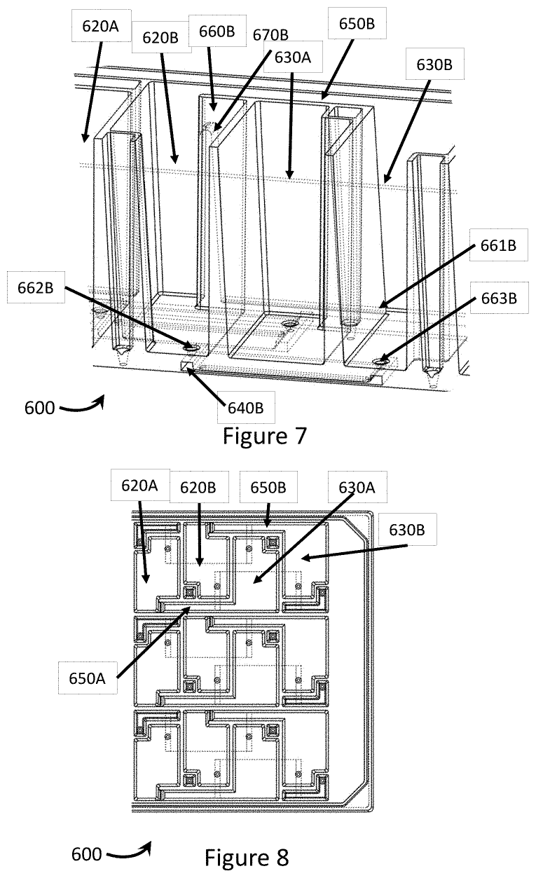

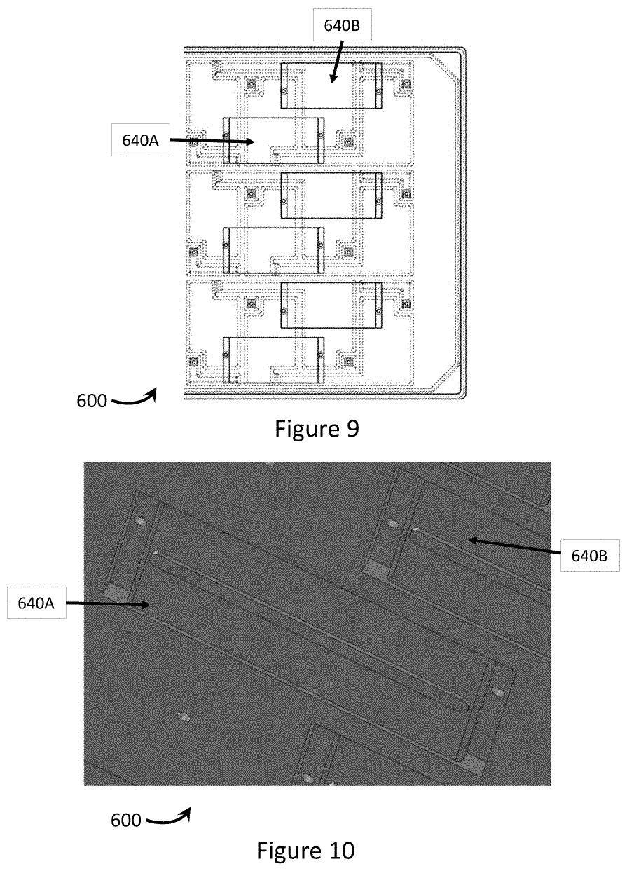

FIG. 7 is a partial cutaway orthogonal view of a segment of the cell culture system of FIG. 6 and FIG. 8 is a top perspective view of the cell culture system of FIG. 6. FIG. 9 is a partial cutaway bottom perspective view of the cell culture system 600 and FIG. 10 is a partial cutaway bottom perspective view of the cell culture system 600, where the position of the cutaway is below the cutaway for FIG. 9. Elements of the cell culture system 600 are obscured from view in FIGS. 7 to 10 by other components of the cell culture system 600.

The following discussion of the cell culture system 600 focuses on the features of one of the recirculation loops, namely recirculation loop 610B, for the sake of clarity. It is understood that the features of recirculation loop 610B are substantively similar to the features of each of the recirculation loops 610.

Recirculation loop 610B includes a supply reservoir 620B, an effluent reservoir 630B, a cell culture chamber 640B, and a bypass channel 650B. In one embodiment, the system 600 may include a removable cover to reduce or limit contamination and/or fluid evaporation. The supply reservoir 620B, the bypass channel 650B, and the effluent reservoir 630B are preferably molded from a single piece of rigid and impermeable material. In other words, a single piece of material defines the position, orientation, and dimensions of each of the supply reservoirs 620, the bypass channels 650 and the effluent reservoirs 630. The fluidic couplings may be openings in the rigid and impermeable material through which fluid may flow. The rigid and impermeable material may be metal, ceramic, plastic, glass, or any other suitable material. While molding the supply reservoir 620B, the bypass channel 650B, and the effluent reservoir 630B from a single piece of rigid material is advantageous, each of the supply reservoir 620B, the bypass channel 650B, and the effluent reservoir 630B could be manufactured as separate pieces and coupled together to produce cell culture system 600.

The supply reservoir 620B is dimensioned and oriented to hold fluid at a predetermined fluid level. The cell culture chamber 640B is fluidically or fluidly coupled between the supply reservoir 620B and the effluent reservoir 630B. The bypass channel 650B is fluidically coupled between the supply reservoir 620B and the effluent reservoir 630B.

When the cell culture apparatus, or system, 600 is in the first position, fluid media held in the supply reservoir 620B flows from the supply reservoir 620B to the effluent reservoir 630B via the cell culture chamber 640B. The flow of fluid media from the supply reservoir 620B to the effluent reservoir 630B via the cell culture chamber 640B lowers the fluid level in the supply reservoir 620B below the predetermined level, and raises the fluid level in the effluent reservoir 630B.

When cell culture system, or apparatus, 600 is in the second position, fluid, or fluid media, flows from the effluent reservoir 630B to the supply reservoir 620B via the bypass channel 650B. The flow of fluid from the effluent reservoir 630B to the supply reservoir 620B via the bypass channel 650B raises the fluid level in the supply reservoir 620B back to the predetermined level and lowers the fluid level in the effluent reservoir 630B to its initial level.

The bypass channel 650B is coupled to the supply reservoir 620B at a supply reservoir and bypass channel coupling position 660B higher than the predetermined fluid level, which reduces the likelihood or prevents fluid from flowing through the bypass channel when cell culture apparatus 600 is in the first position. Each recirculation loop may include a recirculation barrier 670 to define the height of the supply reservoir and bypass channel coupling position 660. The recirculation barrier 670B may be a rigid and impermeable material to prevent or reduce the likelihood of fluid flowing through the bypass channel 650B when cell culture apparatus 600 is in the first position.

The cell culture chamber 640B is positioned below the supply reservoir 620B and below the effluent reservoir 630B, which allows the cell culture chamber 640B to remain filled with fluid when cell culture apparatus 600 is in the first position and when cell culture apparatus 600 is in the second position.

The cell culture chamber 640B, the fluidic coupling between the supply reservoir and the cell culture chamber 662B, and the fluidic coupling between the effluent reservoir and the cell culture chamber 663B define a first flow path with a first flow resistance. The bypass channel 650B, the fluidic coupling between the effluent reservoir and the bypass channel 661B, and the fluidic coupling between the supply reservoir and the bypass channel 660B define a second flow path with a second flow resistance in one embodiment. In one embodiment, the first flow resistance is greater than the second flow resistance. As discussed above, when cell culture apparatus 600 is in the second position, fluid may flow through both the bypass channel 650B and back-flow through the cell culture chamber 640B. A greater flow resistance yields a lower flow rate when fluid flows through a flow path. When the first flow resistance is greater than the second flow resistance, the flow rate through the bypass channel 650B will be greater than the back-flow rate through the cell culture chamber 640B when the cell culture apparatus 600 is in the second position. A greater flow resistance is typically associated with a narrower channel, and/or narrower fluidic couplings between the channel and the supply reservoir and/or effluent reservoir.

A greater rate of flow through the bypass channel 650B is advantageous since this reduces the amount of time that cells growing in the cell culture chamber 640B experience back-flow and ensures that cells growing in the cell culture chamber 640B experience predominantly forward flow. In other words, a greater rate of flow through the bypass channel 650B may enable unidirectional flow through the cell culture chamber 640B. The fluidic couplings between the reservoirs and channels of cell culture apparatus 600 are openings in the rigid material rather than valves or other couplings that require or include moving parts. Since fluid flow is driven by a positioning of the cell culture apparatus 600, recirculating flow in the cell culture apparatus 600 may therefore be achieved without employing moving parts within the cell culture apparatus 600.

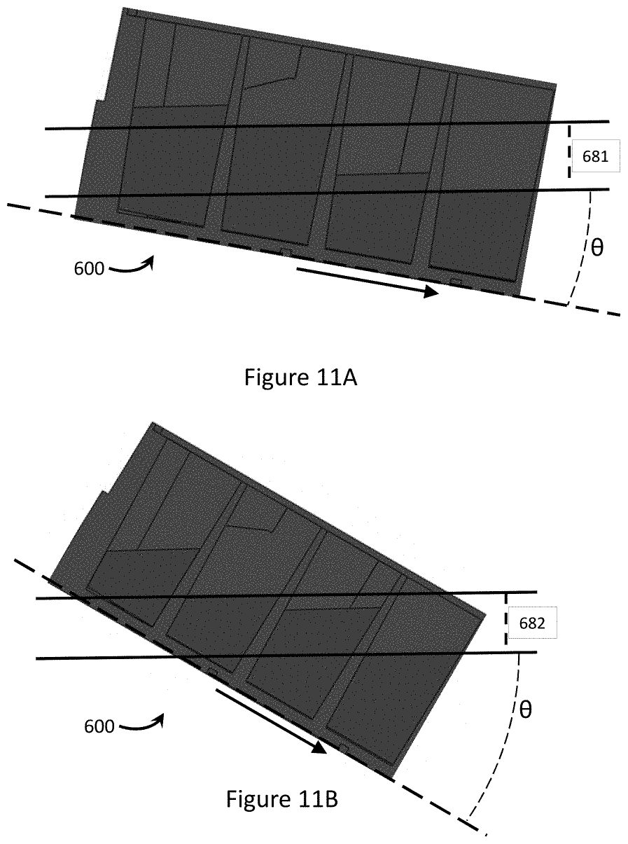

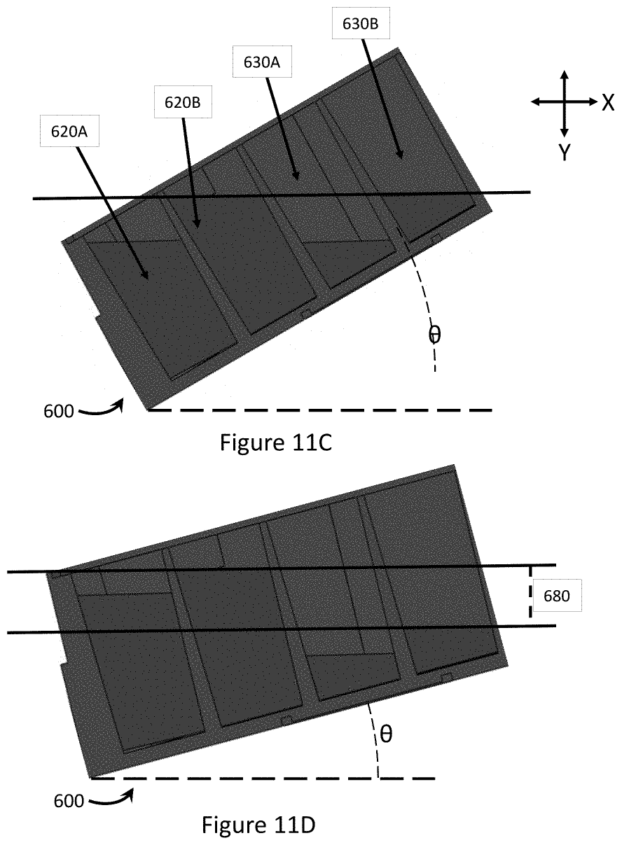

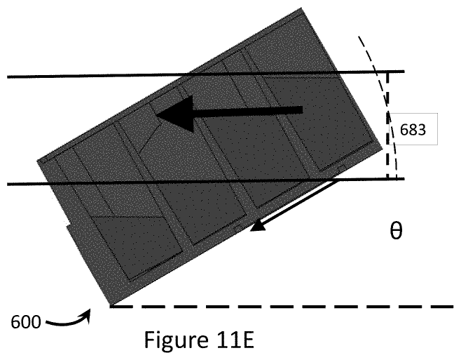

FIGS. 11A to 11E are cross-sectional diagrams of a segment of the cell culture apparatus 600 at various tilt angles containing fluid at various fluid levels. The cross section of cell culture apparatus 600 was taken through the X-Y plane as shown in FIGS. 11A to 11E and a Z-axis (not shown) perpendicular to the X-Y plane. The following discussion of the cell culture apparatus 600 focuses on recirculating loop 610B but it will be appreciated that the movement of fluid caused by positioning, as discussed below, applies to recirculation loops generally.

FIG. 11A shows the cell culture apparatus 600 in an intermediate forward flow position. In other words, after the cell culture apparatus 600 has been tilted to increase the tilt angle of the cell culture apparatus 600 from a neutral position. The neutral position may be seen as a position at which the height of the fluid in the supply reservoir and the height of the fluid in the effluent reservoir are “equal”. The angle at which this occurs will be dependent on the apparatus geometry and the amount of fluid in the system. As can be seen, tilting of the cell culture apparatus clockwise by a tilt angle Θ generates a pressure head 681 in the supply reservoir 620B.

In one embodiment, (and for explanation purposes only), a positive tilt angle may be defined as a tilt angle that positions the supply reservoir 620B above the effluent reservoir 630B, while a negative tilt angle may be defined as an angle that positions the effluent reservoir 630B above the supply reservoir 620B. As is understood, the designation of one tilt direction as being positive while the opposite tilt direction being a negative is arbitrary and does not impact the design, construction, or use of cell culture apparatus 600, but may be used to clarify directionality when discussing tilting of cell culture apparatus 600.

For cell culture systems including multiple recirculation loops or cell culture apparatus, it may be advantageous to align all of the recirculation loops in the same direction, which allows tilting of the apparatus to have the same effect on each recirculation loop.

FIG. 11B shows the cell culture apparatus 600 in State 2. In other words, after the cell culture apparatus 600 has been tilted to the forward flow position. In a preferred embodiment, the tilt angle Θ is continuously changed to maintain a constant pressure head 682 between the supply reservoir 620B and the effluent reservoir 630B.

FIG. 11C shows the cell culture apparatus 600 in the second, or return flow position. As can be seen in FIG. 11c, the cell culture apparatus is approximately preferably between 30 and 40 degrees, and more specifically, is preferred to be about 35 degrees “above” the horizontal.

FIG. 11D shows the cell culture apparatus 600 in an intermediate forward flow position. In other words, after the cell culture apparatus 600 has been tilted to increase the tilt angle of the cell culture apparatus 600 relative to the tilt angle of the cell culture apparatus 600 in FIG. 110. Tilting cell culture apparatus 600 to the intermediate return flow position raises the position of the supply reservoir 620B relative to the position of effluent reservoir 630B thereby raising the fluid level in the supply reservoir 620B relative to the fluid level in the effluent reservoir 630B and generating a pressure head 680 in the supply reservoir 620B. In a preferred embodiment, the pressure head 680 is the same as pressure heads 681 and 682. As discussed above, the pressure head in the supply reservoir 620B drives flow from the supply reservoir 620B to the effluent reservoir 630B via cell culture chamber 640B, thereby reducing the pressure head in the supply reservoir 620B. The reduction in pressure head, with the resulting reduction in flow rate through cell culture chamber 640B, may be undesirable.

Alternatively, the cell culture apparatus 600 could be tilted from the second position directly to the first position, which would result in a pressure head larger than pressure heads 680, 681, and 682. Alternatively, the cell culture apparatus 600 could be tilted continuously from the second position to the first position to maintain a constant pressure head.

FIG. 11E shows the cell culture apparatus 600 in State 4 after the cell culture apparatus 600 has been tilted to decrease the tilt angle of the cell culture apparatus 600 relative to the tilt angle of the cell culture apparatus 600 in FIG. 11B. In other words, FIG. 11E shows the cell culture apparatus 600 after the cell culture apparatus 600 has been tilted from the first position to the second position. Tilting the cell culture apparatus 600 to the second position from the first position raises the effluent reservoir 630B relative to the supply reservoir 620B, raising the fluid level in the effluent reservoir 630B relative to the fluid level in the supply reservoir 620B and generating pressure head 683 in supply reservoir 620B.

Pressure head 683 drives fluid flow from the effluent reservoir 630B to the supply reservoir 620B via both the cell culture chamber 640B and the bypass channel 650B. As the bypass channel 650B has a lower flow resistance than the cell culture chamber 640B, the flow rate through bypass channel 650B is higher than the flow rate through the cell culture chamber 640B such that a majority of the fluid media therefore flows through the bypass channel 650B.

Engineering of cell culture apparatus 600 allows control over the fluid flow rates through the cell culture area 640B. In general, there are two design elements that impact the fluid flow rate: the fluid pressure head and the channel dimensions. The greater the vertical difference between the height of the supply reservoir fluid level and the effluent reservoir fluid level, the faster the fluid will flow. As well, the larger the dimensions of the cell culture chamber and/or the bypass channel, the faster the fluid media will flow. In an alternative embodiment, the position of the cell culture apparatus 600 by the tilting mechanism could be continuously varied by the mechanism, to provide a time-variable or constant fluid flow rate.

In some embodiments, the cell culture chamber contains a porous membrane to define a secondary cell culture chamber. In some embodiments, the construction materials and physical dimensions of cell culture apparatus 600 are specified so as to permit imaging of the cell culture chamber. In some embodiments, sensors are embedded in the reservoirs, channels, and/or chambers in order to monitor the fluid and/or the cells.

Creating recirculating flow in multiple parallel cell culture chambers using a single mechanical actuator may be beneficial as compared to non-recirculating flow. In many cases, the fluid or chemicals in the fluid that is used to culture cells may be expensive or only available in limited quantities. By recirculating the media within in each culture chamber, the media may be used more efficiently.

Recirculating flow may also be beneficial as compared to reciprocating flow without a bypass channel. In reciprocating flow, the fluid flows through cell culture chamber from the supply reservoir to the effluent reservoir and then back again via the same fluid path. Reciprocating flow differs from in vivo conditions, where blood normally flows in one direction only, and may result in undesirable changes to the behaviour of cultured cells.

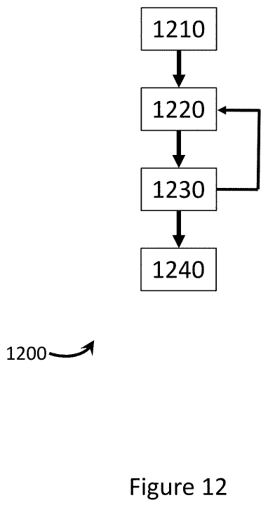

FIG. 12 shows a flow diagram for a method 1200 of recirculating fluid media through a cell culture chamber.

At 1210, an apparatus for culturing cells is provided. The apparatus may include a supply reservoir to hold a fluid at a predetermined fluid level, an effluent reservoir, a cell culture chamber for hosting cells, the cell culture chamber fluidically coupled between the supply reservoir and the effluent reservoir, and a bypass channel fluidically coupled between the supply reservoir and the effluent reservoir.

At 1220, the apparatus is positioned in a first position to flow the fluid media from the supply reservoir to the effluent reservoir via the cell culture chamber with a first flow rate.

Positioning the apparatus in a first position may include raising the position of the supply reservoir relative to the position of the effluent reservoir to create a pressure head in the supply reservoir. The pressure head forms when the fluid level of a fluid in a reservoir is higher than the fluid level in another reservoir to which the former reservoir is fluidically coupled. The pressure head drives fluid flow due to the force of gravity acting on the fluid in the reservoir, and may be measured in millimeters. For example, if the fluid level in the supply reservoir is at the same height as the fluid level in the effluent reservoir, there is no pressure head in either the supply reservoir or the effluent reservoir. A larger pressure head exerts a greater pressure on the fluid and drives faster fluid flow.

As fluid flows from the supply reservoir to the effluent reservoir due to the presence of a pressure head, the fluid level in the supply reservoir decreases and the fluid level in the effluent reservoir increases, thereby decreasing the magnitude of the pressure head. In other words, fluid flowing due to a pressure head decreases the magnitude of the pressure head over time. Since the fluid flow rate depends on the magnitude of the pressure head, positioning the apparatus in one position to generate a pressure head will result in a fluid flow rate that decreases over time. The pressure head in the supply reservoir may be in the range of 0-10 mm.

At 1230, the apparatus is positioned in a second position to flow the fluid media from the effluent reservoir to the supply reservoir, such as via the bypass channel. This is preferably performed at a second flow rate, the second flow rate higher than the first flow rate. Positioning the apparatus in a second position may include raising the position of the effluent reservoir relative to the position of the supply reservoir to create a pressure head in the effluent reservoir.

At 1240, the tilting performed in 1220 and 1230 are repeated to recirculate the fluid media through the cell culture chamber until there is no need for any more fluid recirculation. In other words, the tilting performed in 1220 and 1230 may be repeated until a desired amount of flow of fluid through the cell culture has been achieved, or until flow has been maintained for a desired amount of time.

As discussed above, the flow rate through the cell culture chamber is at least partially determined by the dimensions of the cell culture chamber, and may also be affected by the dimensions of the fluidic couplings between the cell culture chamber, the supply reservoir, and the effluent reservoir. Likewise, the flow rate through the bypass channel is at least partially determined by the dimensions of the bypass channel, and may also be affected by the dimensions of the fluidic couplings between the bypass channel, the supply reservoir, and the effluent reservoir. Narrower dimensions reduce the flow rate, while broader dimensions increase the flow rate. Non-exclusive examples of narrower dimensions include: the bypass channel having a larger cross-sectional area than the cell culture chamber, the couplings into/out of the bypass channel being larger than the couplings into/out of the cell culture chamber. Larger couplings includes couplings with a larger effective area, for example a coupling filled with porous material (e.g. a glass frit) has a smaller effective area than an equivalent un-filled coupling.

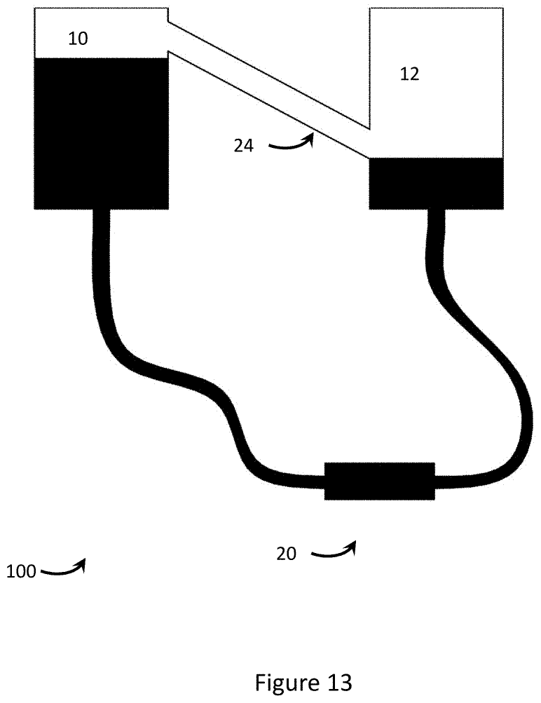

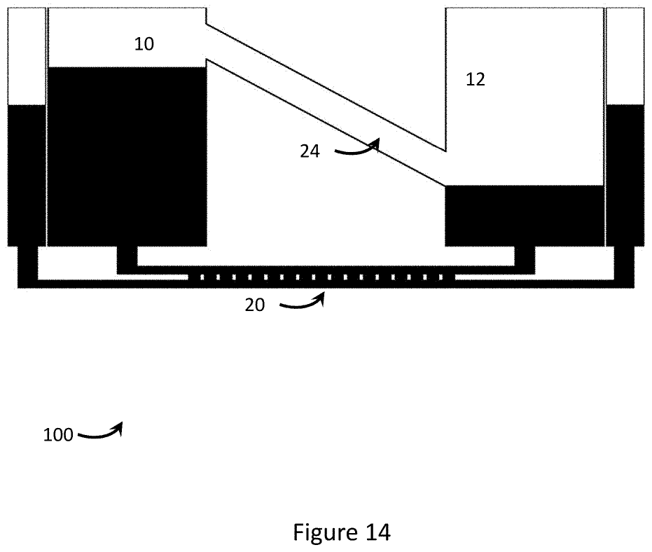

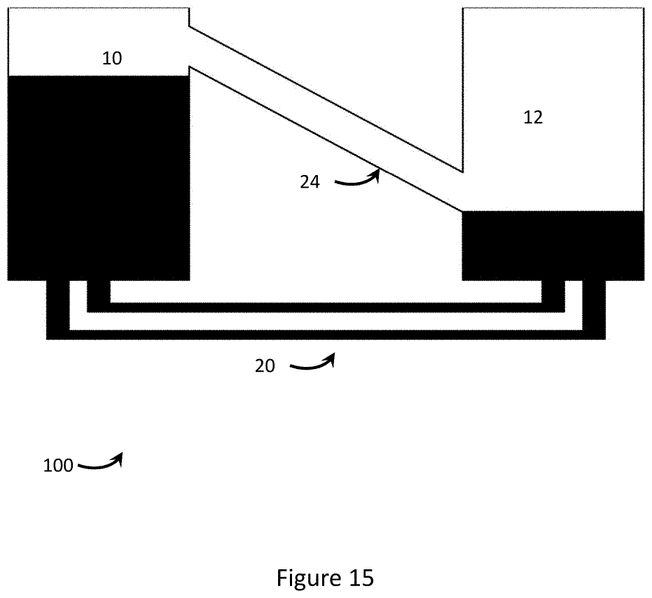

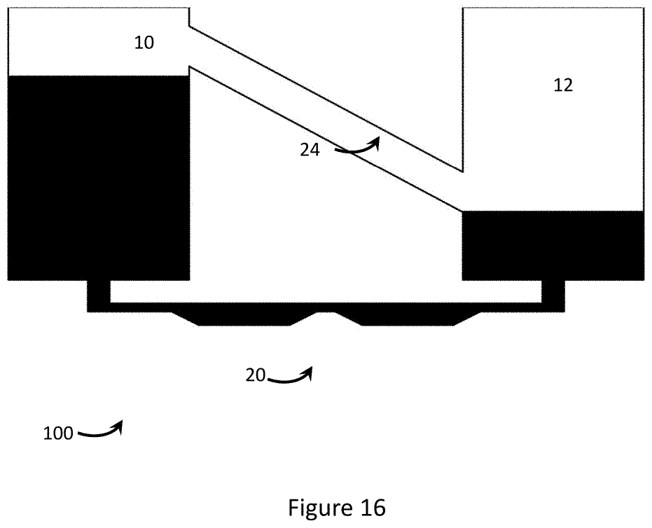

Turning to FIGS. 13 to 16, further embodiments of cell culture apparatus are shown. Each of the apparatus include the supply reservoir, the effluent reservoir and the bypass channel and a different cell culture chamber. In FIG. 13, the apparatus 100 has a remote cell culture chamber 20. In FIG. 14, the apparatus 100 includes two cell culture chambers with a single porous membrane. In FIG. 15, the apparatus 100 includes a cell culture chamber with two parallel channels and in FIG. 16, the apparatus 100 includes a pair of cell culture chamber in series with each other.

While the fluid is flowing through the apparatus 600, the apparatus may be imaged, or more specifically, the cell culture chamber may be imaged. In one embodiment, this is performed using inverted microscopy where a lower surface of the cell culture chamber is fashioned from a thin material that is transparent at the appropriate wavelengths. For example the bottom of the chamber could be made from 0.2 mm glass so as to be optically similar to a microscope coverslip. The design of the overall apparatus could be such that the footprint of the apparatus conforms to a standard Society for Biomolecular Screening (SBS) plate format in order to be compatible with existing imaging equipment.

It will be appreciated that the above description relates to the preferred embodiments by way of example only. Many variations on the disclosure will be obvious to those knowledgeable in the field, and such obvious variations are within the scope of the disclosure as described, whether or not expressly described. For example, the supply reservoir and/or the effluent reservoir may be open-topped or enclosed. The effluent reservoir may be aligned with the supply reservoir at various angles relative to the bypass channel and the cell culture channel. The bypass channel may be sloped or flat, open-topped or enclosed. A common application of the disclosure will be for culturing cells, with the cells contained in the cell culture chamber, but the disclosure is not limited to that.

In the preceding description, for purposes of explanation, numerous details are set forth in order to provide a thorough understanding of the embodiments. However, it will be apparent to one skilled in the art that these specific details are not required. The above-described embodiments are intended to be examples only. Alterations, modifications and variations can be effected to the particular embodiments by those of skill in the art without departing from the scope, which is defined solely by the claims appended hereto.

1. An apparatus for culturing cells comprising:
a supply reservoir;
an effluent reservoir;
a cell culture chamber for hosting cells, the cell culture chamber fluidically coupled between the supply reservoir and the effluent reservoir; and
a bypass channel fluidically coupled between the supply reservoir and the effluent reservoir;
wherein when the apparatus is in a first position, fluid held in the supply reservoir flows to the effluent reservoir via the cell culture chamber and when the apparatus is in a second position, fluid flows from the effluent reservoir to the supply reservoir via the bypass channel.

2. The apparatus of claim 1 wherein the supply reservoir, the effluent reservoir, the cell culture chamber and the bypass chamber as formed by a single piece of material.

3. The apparatus of claim 2 wherein the single piece of material is rigid and porous.

4. The apparatus of claim 2 wherein the single piece of material is metal, ceramic, plastic or glass.

5. The apparatus of claim 1 wherein in the second position, fluid flows from the effluent reservoir to the supply reservoir via the cell culture chamber.

6. The apparatus of claim 1 wherein the bypass channel is coupled to the supply reservoir at a supply reservoir and bypass channel coupling position higher than the fluid level when the apparatus is in the first position.

7. The apparatus of claim 6, further comprising a recirculation barrier to define a height of the supply reservoir and bypass channel coupling position.

8. The apparatus of claim 6 wherein the bypass channel is coupled to the effluent reservoir at a effluent reservoir and bypass channel coupling position lower than the supply reservoir and bypass channel coupling position.

9. The apparatus of claim 7 wherein the cell culture chamber is coupled to the supply reservoir and the effluent reservoir at positions beneath the supply reservoir and bypass channel coupling position and effluent reservoir and bypass channel coupling position.

10. The apparatus of claim 1, wherein the cell culture chamber is positioned below the supply reservoir and below the effluent reservoir.

11. The apparatus of claim 1 wherein the cell culture chamber comprises at least two channels.

12. The apparatus of claim 11 wherein the at least two channels are parallel to each other.

13. The apparatus of claim 11 wherein the at least two channels are connected via a porous membrane.

14. The apparatus of claim 1 wherein the cell culture chamber comprises at least two chambers.

15. The apparatus of claim 14 wherein the at least two chambers are in series with each other.

16. The apparatus of claim 1 wherein the cell culture chamber is remote from the supply reservoir and the effluent reservoir.

17. A system for culturing cells comprising:
a set of cell culture apparatus, each of the cell culture apparatus including:
a supply reservoir;
an effluent reservoir;
a cell culture chamber for hosting cells, the cell culture chamber fluidically coupled between the supply reservoir and the effluent reservoir; and
a bypass channel fluidically coupled between the supply reservoir and the effluent reservoir;
wherein when the system is in a first position, fluid held in the supply reservoir of each of the set of cell culture apparatus flows to the effluent reservoir via the cell culture chamber and when the system is in a second position, fluid flows from the effluent reservoir of each of the cull culture apparatus to the supply reservoir via the bypass channel.

18. The system of claim 17 wherein the set of cell culture apparatus are spatially segregated from each other.

19. The system of claim 17 wherein the set of cell culture apparatus are interleaved with each other.

20. The system of claim 17 wherein the set of cell culture apparatus is formed by a single piece of material.
